(19) United States
(12) Patent Application Publication
(10) Pub. No.: US 2023/0320907 A1
(43) Pub. Date: Oct. 12, 2023

(54) NIPPLE COVER

(71) Applicant: Yoon Young NA, Goyang-si (KR)

(72) Inventor: Yoon Young NA, Goyang-si (KR)

(21) Appl. No.: 17/981,974

(22) Filed: Nov. 7, 2022

(30) Foreign Application Priority Data
Apr. 6, 2022 (KR) 10-2022-0042733

(51) Int. Cl. A61F 13/14; A61F 13/02

(52) U.S. Cl. CPC A61F 13/14; A61F 13/0253

(57) ABSTRACT

Provided is a nipple cover pressing and covering a nipple and the surrounding skin to prevent nipple protrusion. The nipple cover is thin, light, and soft and reduces a foreign body sensation when attached to the skin. The nipple cover includes a cover sheet and an adhesive layer. The cover sheet is made of a mesh-type fabric to enhance the pressing force and to increase the coverage of the nipple cover. The adhesive layer is made of a silicone gel that minimizes skin irritation and which enables the reuse of the nipple cover by cleansing.

### BACKGROUND OF THE DISCLOSURE

### 1. Field of the Disclosure

The present disclosure relates to a nipple cover pressing and covering a nipple to prevent nipple protrusion by covering skin around the nipple. The nipple cover is thin, light, and soft and reduces a foreign body sensation when attached to the skin. In addition, the nipple cover includes a cover sheet and an adhesive layer. The cover sheet is made of a mesh-type fabric to enhancing the pressing force and to increase the coverage of the nipple cover. Furthermore, an adhesive layer is made of a silicone gel that minimizes skin irritation and which enables the reuse of the nipple cover by cleansing.

### 2. Description of the Related Art

Generally, women wear brassieres to prevent breasts from sagging and look stylish by enhancing the appearance of breasts. However, brassieres tighten the chest and thus cause discomfort, which is unhealthy. In addition, in the summertime when people feel hot and sweaty, it's even further inconvenient to wear brassieres.

For these reasons, many women are reluctant to wear the brassieres. However, without the brassieres, nipples may show through a top, which looks unfashionable and unattractive.

On the other hand, such problems are not just for women. Even when men wear tight t-shirts, nipples may also show through, which looks unattractive.

In order to solve the problem of the nipple showing through the top, Registered Utility Model No. 20-0322617 on “NIPPLE COVER”, Registered Utility Model No. 20-0485610 on “PACKAGE OF NIPPLE-COVERING BAND”, and the like have been proposed in the related art.

“NIPPLE COVER” of Registered Utility Model No. 20-0322617 is composed of a main body and an adhesive member. The main body covers a nipple and skin around the nipple, and the adhesive member is formed on an inner surface of the main body and adheres to the skin. In addition, “PACKAGE OF NIPPLE-COVERING BAND” of Registered Utility Model No. 20-0485610 is composed of a nipple-covering band and an adhesive layer. The nipple-covering band covers the nipple and skin around the nipple, and the adhesive layer is formed on an inner surface of the nipple-covering band.

In the above related art, the main body is made of polyurethane, and the nipple-covering band is made of a synthetic resin, such as PVC.

In addition, in the related art, the main body and the nipple-covering band are thick and heavy. As a result, the foreign body sensation of skin attached to the nipple cover may be worsened, thereby causing discomfort. In addition, due to a rough surface of the nipple cover, the outer surface being exposed feels significantly different from the soft skin around the nipple. Furthermore, the nipple and skin around the nipple being in contact with the inner surface of the nipple cover are irritated. Moreover, the nipple pressing force of the main body and the nipple-covering band and the lasting effect of the above pressing force are weak. As a result, the efficiency in preventing the protrusion of the nipple is not so high. In addition, the main body and the nipple-covering band are difficult to be blended into the skin around the nipple, so coverage of the nipple cover is low.

In the related art, the adhesion of the adhesive member and the adhesive layer remarkably decreases after a single use, which makes them difficult to be reused. Thus, as the nipple covers in the related art are disposable products, cost burdens are high.

### SUMMARY OF THE INVENTION

The present disclosure is proposed to solve the above problems of the nipple covers in the related art. An objective of the present disclosure is to provide a nipple cover which reduces a foreign body sensation and is wearable. The nipple cover prevents the nipple from protruding without exception, has excellent nipple coverage, and minimizes skin irritation.

In addition, another objective of the present disclosure is to provide a nipple cover in which an adhesive layer is prevented from folding and adhering to itself when removed from a release film. The nipple cover is easily and quickly attachable without wrinkles and curves when attached to the nipple and skin around the nipple.

In order to achieve the above objectives, a nipple cover, according to the present disclosure, includes an adhesive layer adhering to a nipple and skin around the nipple, a cover sheet attached to an outer surface of the adhesive sheet, and a flexible elastomer sheet attached to an outer surface of the cover sheet. The cover sheet is made of a mesh-type fabric to firmly press the nipple.

In addition, the nipple cover further includes a raschel sheet attached to an inner surface of the adhesive sheet to press the nipple and to prevent the adhesive sheet from adhering to an object.

In addition, the adhesive layer is made of a silicone gel reusable through cleansing.

In addition, the adhesive sheet includes an anti-folding member so that the nipple cover remains unfolded, and the anti-folding member is made of a material soluble at body temperature.

According to the present disclosure configured as described above, a nipple cover includes a cover sheet, an elastomer sheet, and an adhesive layer. The cover sheet is made of a mesh-type fabric to firmly press a nipple and skin around the nipple so that the nipple is prevented from protruding without exception. In addition, the cover sheet allows the nipple cover to be blended into the skin around the nipples to which the nipple cover is attached. As a result, it is unnoticeable that the nipple cover is attached to the skin, thereby increasing the coverage of the nipple cover. The elastomer sheet has a surface which provides a soft skin-like feel and has excellent flexibility, thereby enabling the skin to move freely. In addition, the adhesive layer is made of a medical-grade silicone gel, thereby minimizing skin irritation. Furthermore, the adhesive layer is reusable through cleansing, and thus reduces cost burden. A raschel sheet can be additionally used, as needed, to prevent skin irritation and an uncomfortable feeling from the adhesive layer and to increase the nipple coverage. As a result, the nipple cover is made into a product which is thin, light, and soft overall, reduces a foreign body sensation, and has an excellent wearability. In addition, when removed from a release film, the nipple cover remains unfolded and thus is easily attached to the skin around the nipple without wrinkles and curves. Furthermore, once the nipple cover is attached to the skin, an anti-folding member is deactivated, and thus the nipple cover is easily deformable. As a result, the nipple cover can provide a soft feel and enables the skin to move freely. Hence, the present disclosure is highly useful for industrial development.

### BRIEF DESCRIPTION OF THE DRAWINGS

FIG. 1 is a prospective view and a cross-sectional view of a nipple cover according to the present disclosure;

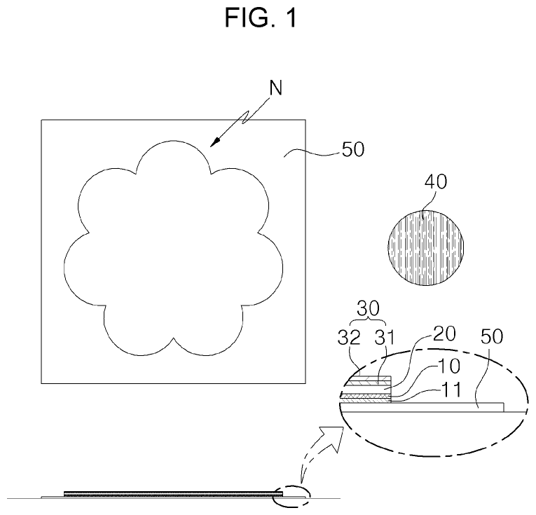

FIG. 2 is an exploded view of a nipple cover; and

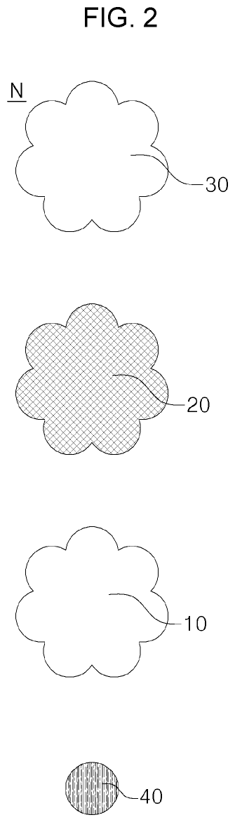

FIG. 3 is a diagram illustrating an adhesive sheet including an anti-folding member.

### DESCRIPTION OF THE PREFERRED EMBODIMENTS

As shown in the drawings, a nipple cover N, according to the present disclosure, includes an adhesive sheet 10, a cover sheet 20, and an elastomer sheet 30. In addition, the nipple cover N may further include a raschel sheet 40, as needed. Furthermore, the nipple cover N includes a release film 50 to protect an adhesive layer 11 in the adhesive sheet 10 during the production and distribution of the nipple cover N.

The adhesive sheet 10 covers a nipple and skin around the nipple.

The adhesive layer 11 is formed on an inner surface of the adhesive sheet 10 to adhere to the nipple and skin around the nipple. The adhesive layer 11 has adhesion so that the nipple cover does not come off easily from the skin to which the adhesive layer 11 adheres.

The adhesive layer 11 is made of a medical-grade silicone gel. Preferably, the adhesive layer 11 minimizes skin irritation when in contact with the sensitive nipple and skin around the nipple.

In addition, the silicone gel can be reused through cleansing. Therefore, the nipple cover N can be used multiple times, which can innovatively reduce cost burden. Generally, once the nipple cover is used, foreign substances are attached to the adhesive layer. As a result, the adhesion of the adhesive layer decreases, and thus it is difficult to reuse the nipple cover. However, foreign substances attached to the adhesive layer 11 made of the silicone gel can be removed by cleansing. In addition, since a substantial degree of the adhesion is preserved even after being cleansed, the nipple cover N can be reused. As a result of experiments, the adhesive layer 11 made of the silicone gel can be reused about 5 times through cleansing with ordinary water. Even when the adhesive layer 11 is cleansed with water, the adhesion may decrease due to the remaining foreign substances attached to the adhesive layer 11. In this case, the adhesion can be restored by being cleansed with a dedicated cleaning agent. As a result, when the dedicated cleaning agent is used, the adhesive layer 11 made of the silicone gel can be reused about 20 times.

The adhesive layer 11 of the nipple cover N is cleansed with water or the dedicated cleaning agent and dried. Then, the nipple cover is attached to the release film 50 to be stored and reused as needed.

The adhesive sheet 10 may be made of a nonwoven fabric, a synthetic resin, paper, a composite material, and the like. However, without additionally using the above materials, the adhesive layer 11 itself may be used as the adhesive sheet 10.

The cover sheet 20 is attached to an outer surface of the adhesive sheet 10.

The cover sheet 20 uses a well-structured mesh-type fabric to hold and firmly presses the skin including the nipple and areola. As a result, the cover sheet 20 prevents the nipple from protruding and allows the nipple cover N to be blended into the skin around the nipple, thereby increasing the coverage of the nipple cover. Thus, it is unnoticeable that the nipple cover N is attached to the skin.

The elastomer sheet 30 is attached to an outer surface of the cover sheet 20.

The elastomer sheet 30 is highly flexible to enable the skin to move freely in daily life. In addition, the elastomer sheet 30 has a surface that provides a soft skin-like feel.

The elastomer sheet 30 includes a flexible elastomer layer 31 having excellent elasticity, which is made of silicone, and a skin-like layer 32 that provides a soft skin-like feel, which is made of a PU resin.

The raschel sheet 40 may be attached to an inner surface of the adhesive sheet 10, as selected.

Those who do not like the feeling of the adhesive layer 11 in the adhesive sheet 10 adhering to the skin, or whose skin is irritated by the adhesion of the adhesive layer 11, may use the raschel sheet 40 covering the sensitive nipple and areola by attaching the raschel sheet 40 to a central portion of the adhesive sheet 10.

The raschel sheet 40 has a fine air-permeable structure and is made of a soft and sturdy nonwoven fabric.

The raschel sheet 40 prevents the adhesive layer 11 from adhering to an object. In addition, the raschel sheet 40 presses the nipple and areola, to which the raschel sheet 40 is attached, to prevent the nipple from protruding once more and additionally increase the nipple coverage.

The release film 50 covers and protect the adhesive layer 11 in the adhesive sheet 10 during the production, transportation before using, and storage. When the nipple cover N is used, the release film 50 is removed from the adhesive sheet 10 so that the adhesive layer 11 can adhere to the skin.

The nipple cover N is soft and easily deformable. As a result, the skin to which the nipple cover N is attached can move freely when the nipple cover N is attached to the nipple and skin around the nipple.

Hence, when removed from the release film 50, the nipple cover N is often likely to be folded inward, so that the adhesive layer 11 in the adhesive sheet 10 adheres to itself.

In addition, when attached to the skin around the nipple, the nipple cover N is required to be randomly stretched by force with fingers, which is inconvenient. Nevertheless, the nipple cover N is likely to be attached with curves in random spots.

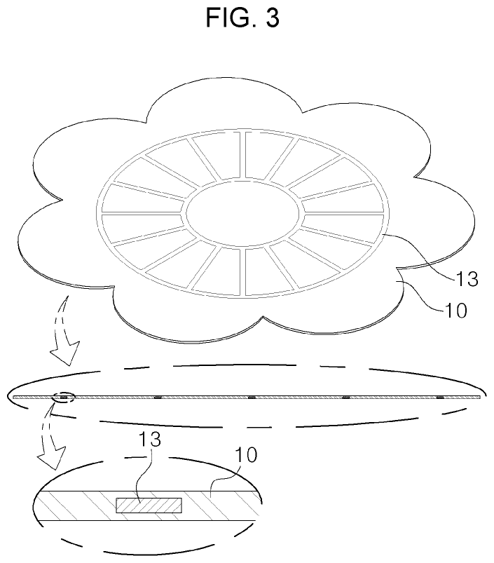

Referring to FIG. 3, in order to solve such problems, the present disclosure provides an anti-folding member 13 included in the adhesive sheet 10. Therefore, when removed from the release film 50, the nipple cover N may remain unfolded. The anti-folding member 13 is included in an internal space of the adhesive sheet 10 and thus unexposed to the outside.

However, in this case, when the nipple cover N remains unfolded even after being attached to the skin around the nipple due to the anti-folding member 13, the nipple cover N may be unable to sufficiently provide a soft feel. In addition, the skin to which the nipple cover N is attached may not move freely.

Therefore, the anti-folding member 13 is made of a material soluble at body temperature so that the anti-folding member 13 is deactivated when the nipple cover N is attached to the skin.

The anti-folding member 13 is made of a material that is a solid at room temperature and melts at about 30° C. to become a liquid. Examples of such a material include gallium, cesium, cocoa butter, and the like.

Referring to FIG. 3, the anti-folding member 13 may be composed of two rings, positioned inward and outward, and connecting lines arranged to connect the two rings.

Thus, by employing the anti-folding member 13, the nipple cover N, according to the present disclosure, remains unfolded when removed from the release film 50. As a result, the nipple cover N can be easily and quickly attached without wrinkles and curves when attached to the nipple and skin around the nipple. In addition, since the anti-folding member 13 is deactivated once the nipple cover N is completely attached to the skin, the nipple cover N is easily deformable, thereby providing a soft feel and enabling the skin to move freely.

Although in the following description of the preferred embodiments of the present disclosure, the nipple cover in the form of a predetermined form with a predetermined structure has been disclosed with reference to the accompanying drawings, those skilled in the art will appreciate that various alternatives, modifications, additions, and substitutions are possible that may be included within the scope and spirit of the present disclosure as defined by the appended claims.

What is claimed is:

1. A nipple cover comprising:
an adhesive sheet comprising an adhesive layer adhering to a nipple and skin around the nipple;
a cover sheet attached to an outer surface of the adhesive sheet; and
a flexible elastomer sheet attached to an outer surface of the cover sheet,
wherein the cover sheet is made of a mesh-type fabric to firmly press the nipple.

2. The nipple cover of claim 1, further comprising a raschel sheet attached to an inner surface of the adhesive sheet to press the nipple and to prevent the adhesive sheet from adhering to an object.

3. The nipple cover of claim 1, wherein the adhesive layer is made of a silicone gel reusable through cleansing.

4. The nipple cover of claim 1, wherein the adhesive sheet comprises an anti-folding member so that the nipple cover remains unfolded, and
the anti-folding member is made of a material soluble at body temperature.
